(19) United States
(12) Patent Application Publication
(10) Pub. No.: US 2024/0217959 A1
(43) Pub. Date: Jul. 4, 2024

(54) New (Homo)Piperidinyl Heterocycles as Sigma Ligands

(71) Applicant: ACONDICIONAMIENTO TARRASENSE, TERRASSA BARCELONA) (ES)

(72) Inventors: José Luis DÍAZ-FERNÁNDEZ, MANRESA (BARCELONA) (ES); Carmen ALMANSA-ROSALES, BARCELONA (ES); Ute CHRISTMANN, VILADECANS (BARCELONA) (ES)

(73) Assignee: ACONDICIONAMIENTO TARRASENSE, TERRASSA BARCELONA) (ES)

(21) Appl. No.: 18/554,195

(22) PCT Filed: Apr. 6, 2022
(86) PCT No.: PCT/EP2022/059085

§ 371 Date: Oct. 5, 2023

(30) Foreign Application Priority Data
Apr. 7, 2021 (EP) 21382291.9

(51) Int. Cl. C07D 413/14; A61K 31/506; A61K 31/5355; A61K 31/551; A61K 31/553; C07D 401/14; C07D 491/107

(52) U.S. Cl. CPC C07D 413/14; A61K 31/506; A61K 31/5355; A61K 31/551; A61K 31/553; C07D 401/14; C07D 491/107

(57) ABSTRACT

The present invention relates to new compounds of formula (I) as sigma ligands having a great affinity for sigma receptors, sigma-1 receptor (σ₁) and/or sigma-2 receptor (σ₂). The present invention also refers to the process for the preparation thereof, to compositions comprising them, and to their use as medicaments.

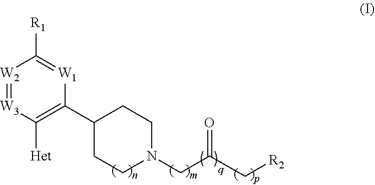

### FIELD OF THE INVENTION

The present invention relates to new compounds of formula (I):

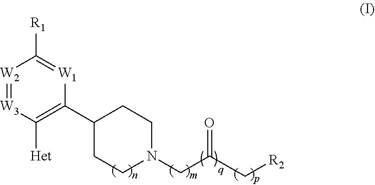

as sigma ligands having a great affinity for sigma receptors, sigma-1 receptor (σ₁) and/or sigma-2 receptor (σ₂). The present invention also refers to the process for the preparation thereof, to compositions comprising them, and to their use as medicaments.

### BACKGROUND OF THE INVENTION

The search for new therapeutic agents has been greatly aided in recent years by better understanding of the structure of proteins and other biomolecules associated with target diseases. One important class of these proteins are the sigma (σ) receptors, originally discovered in the central nervous system (CNS) of mammals in 1976 and initially related to the dysphoric, hallucinogenic and cardiac stimulant effects of opioids. Subsequent studies established a complete distinction between the o receptors binding sites and the classical opiate receptors. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease [Walker, J. M. et al., Pharmacological Reviews, (1990), 42, 355]. It has been reported that the known sigma receptor ligand rimcazole clinically shows effects in the treatment of psychosis [Snyder, S. H., Largent, B. L., J. Neuropsychiatry, (1989), 1, 7]. The sigma binding sites have preferential affinity for the dextrorotatory isomers of certain opiate benzomorphans, such as (+)-SKF-10047, (+)-cyclazocine, and (+)-pentazocine and also for some narcoleptics such as haloperidol. The sigma receptor has two subtypes that were initially discriminated by stereoselective isomers of these pharmacoactive drugs. (+)-SKF-10047 has nanomolar affinity for the sigma-1 (σ₁) site, and has micromolar affinity for the sigma-2 (σ₂) site. Haloperidol has similar affinities for both subtypes.

The σ₁ receptor is expressed in numerous adult mammal tissues (e.g. central nervous system, ovary, testicle, placenta, adrenal gland, spleen, liver, kidney, gastrointestinal tract) as well as in embryo development from its earliest stages, and is apparently involved in a large number of physiological functions. Its high affinity for various pharmaceuticals has been described, such as for (+)-SKF-10047, (+)-pentazocine, haloperidol and rimcazole, among others, known ligands with analgesic, anxiolytic, antidepressive, antiamnesic, antipsychotic and neuroprotective activity. Hence, the σ₁ receptor has possible physiological roles in processes related to analgesia, anxiety, addiction, amnesia, depression, schizophrenia, stress, neuroprotection and psychosis [Walker, J. M. et al., Pharmacological Reviews, (1990), 42, 355; Kaiser, C. et al., Neurotransmissions, (1991), 7 (1), 1-5; Bowen, W. D., Pharmaceutica Acta Helvetiae, (2000), 74, 211-218].

The σ₁ receptor is a ligand-regulated chaperone of 223 amino acids and 25 kDa cloned in 1996 and crystallized twenty years later [Hanner, M. et al., Proc. Natl. Acad. Sci. USA, (1996), 93, 8072-8077; Su, T. P. et al., Trends Pharmacol. Sci., (2010), 31, 557-566; Schmidt, H. R. et al., Nature, (2016), 532, 527-530]. Residing primarily in the interface between the endoplasmic reticulum (ER) and mitochondrion, referred to as the mitochondria-associated membrane (MAM), it can translocate to the plasma membrane or ER-membrane and regulate the activity of other proteins by modulating N-methyl-D-aspartic (NMDA) receptors and several ion channels [Monnet, F. P. et al., Eur. J. Pharmacol., (1990), 179, 441-445; Cheng, Z. X. et al., Exp. Neurol., (2010), 210, 128-136]. Owing to the role played by the σ₁R in modulating pain-related hypersensitivity and sensitization phenomena, σ₁R antagonists have been also proposed for the treatment of neuropathic pain [Drews, E. et al., Pain, 2009, 145, 269-270; De la Puente, B. et al., Pain (2009), 145, 294-303; Díaz, J. L. et al., J. Med. Chem., (2012), 55, 8211-8224; Romero et al., Brit. J. Pharm., (2012), 166, 2289-2306; Merlos, M. et al., Adv. Exp. Med. Biol., (2017), 964, 85-107]. Additionally, the σ₁ receptor has been known to modulate opioid analgesia, and the relationship between the μ-opioid and σ₁ receptors has been shown to involve direct physical interaction, which explains why σ₁ receptor antagonists enhance the antinociceptive effect of opioids without increasing their adverse effects [Chien, C. C. et al, J. Pharmacol. Exp. Ther., (1994), 271, 1583-1590; King, M. et al, Eur. J. Pharmacol., (1997), 331, R5-6; Kim, F. J. et al., Mol. Pharmacol., (2010), 77, 695-703; Zamanillo, D. et al., Eur. J. Pharmacol., (2013), 716, 78-93].

The σ₂ receptor was initially identified by radioligand binding as a site with high affinity for di-o-tolylguanidine (DTG) and haloperidol [Hellewell, S. B. et al., Brain Res., (1990), 527, 244-253]. Two decades later, progesterone receptor membrane component 1 (PGRMC1), a cytochrome-related protein that binds directly to heme and regulates lipid and drug metabolism and hormone signaling, was proposed as the complex where resides the σ₂R binding site [Xu, J. et al., Nat. Commun., (2011), 2, 380]. Finally, in 2017, the σ₂R subtype was purified and identified as transmembrane protein-97 (TMEM97), an endoplasmic-reticulum-resident molecule implicated in cholesterol homeostasis due to its association with the lysosomal Niemann-Pick cholesterol transporter type 1 (NPC1) [Alon, A. et al., Proc. Natl. Acad. Sci. USA, (2017), 114, 7160-7165; Ebrahimi-Fakhari, D. et al., Human Molecular Genetics, (2016), 25, 3588-3599]. The role of σ₂ receptor in cholesterol pathways was known since the 1990s and recent studies published by Mach et al. on modulation of trafficking and internalization of LDL by the formation of a ternary complex between LDLR, PGRMC1 and TMEM97, reinforces this association [Moebius, F. F. et al., Trends Pharmacol. Sci., (1997), 18, 67-70; Riad, A. et al., Sci. Rep., (2018), 8, 16845].

σ₂R/TMEM97, previously known also as meningioma-associated protein, MAC30, is expressed in various normal and diseased human tissues and up-regulation in certain tumors and down-regulation in other suggested that this protein played a distinct role in human malignancies. The cloning of σ₂ receptor confirmed its overexpression in epithelial, colorectal, ovarian lung and breast cancers [Moparthi, S. B. et al., Int. J. Oncol., (2007), 30, 91-95; Yan, B. Y. et al., Chemotherapy, (2010), 56, 424-428; Zhao, Z. R.; Chemotherapy, (2011), 57, 394-401; Ding, H. et al., Asian Pac. J. Cancer Prev., (2016), 17, 2705-2710]. σ₂R/TMEM97 has a molecular weight of 18-21.5 kDa and its sequence predicts a four transmembrane domain protein with cytosolic N and C terminal [Hellewell, S. B. et al., Eur. J. Pharmacol. Mol. Pharmacol. Sect., (1994), 268, 9-18]. The potential signal transduction of σ₂ receptor is not yet understood, but it seems to modulate Ca²⁺ and K⁺ channels, and to interact with caspases, epidermal growth factor receptor (EGFR), and with mammalian target of rapamycin, mTOR, signaling pathways [Vilner, B. J. et al., J. Pharmacol. Exp. Ther., (2000), 292, 900-911; Wilke, R. A. et al., J. Biol. Chem., (1999), 274, 18387-18392; Huang, Y.-S. et al., Med. Res. Rev., (2014), 34, 532-566]. These findings would explain the apoptotic effect of some σ₂ ligands by lysosome dysfunction, reactive oxygen species (ROS) production and caspase-dependent events [Ostenfeld, M. S. et al., Autophagy, (2008), 4, 487-499; Hornick, J. R. et al., J. Exp. Clin. Cancer Res., (2012), 31, 41; Zeng, C. et al., Br. J. Cancer, (2012), 106, 693-701; Pati, M. L. et al., BMC Cancer, (2017), 17, 51].

The σ₂ receptor is involved also in dopaminergic transmission, microglia activation, and neuroprotection [Guo, L. et al., Curr. Med. Chem. (2015), 22, 989-1003]. Terada et al. published in 2018 that σ₂ ligands enhance nerve growth factor (NGF)-induced neurite outgrowth in PC12 cells [Terada, K. et al., Plos One, (2018), 13, e0209250]. The σ₂ receptor plays a key role in amyloid β (Aβ)-induced synaptotoxicity, and σ₂ receptor ligands that block the interaction of Aβ oligomers with the σ₂ receptor have been shown to be neuroprotective [Izzo, N. J. et al., Plos One, (2014), 9, e111899]. σ₂ receptor modulators improve cognitive performance in a transgenic mouse model of Alzheimer's disease (AD), and in two mouse traumatic brain injury models, and could also reduce ischemic stroke injury by enhancing glial cell survival, blocking ischemia-induced glial cell activation, and decreasing nitrosative stress [Katnik, C. et al., J. Neurochem., (2016), 139, 497-509; Yi, B. et al., J. Neurochem., (2017), 140, 561-575; Vázquez-Rosa, E. et al., ACS Chem. Neurosci., (2019), 10, 1595-1602]. The σ₂ receptor has been implicated in other neurological disorders as schizophrenia [Harvey, P.D. et al., Schizophrenia Research (2020), 215, 352-356], alcohol abuse [Scott, L. L. et al., Neuropsychopharmacology, (2018), 43, 1867-1875] and pain [Sahn, J. J. et al., ACS Chem. Neurosci., (2017), 8, 1801-1811]. Norbenzomorphan UKH-1114, a σ₂ ligand, relieved mechanical hypersensitivity in the spared nerve injury (SNI) mice model of neuropathic pain, an effect explained by the preferential expression of σ₂R/TMEM97 gene in structures involved in pain such as the dorsal root ganglion (DRG).

The σ₂ receptor requires two acidic groups (Asp29, Asp56) for ligand binding, similar to σ₁R, which requires Asp126 and Glu172. σ₁R and σ₂R might have similarities in their binding sites but not necessarily other structural similarities if their amino acid sequences are compared. As σ₁R, σ₂ receptor interacts with a wide range of signaling proteins, receptors and channels, but the question if σ₂ receptor has a primarily structural or a modulatory activity remains to be answered. Several classes of σ₂ receptor ligands have been developed since Perregaard et al., synthesized Siramesine and indole analogues in 1995 [Perregaard, J. et al., J. Med. Chem., (1995), 38, 1998-2008]: tropanes [Bowen, W. D. et al., Eur. J. Pharmacol., (1995), 278, 257-260], norbenzomorphans [Sahn, J. J. et al., ACS Med. Chem. Lett., (2017), 8, 455-460], tetrahydroisoquinolines [Sun, Y.-T. et al., Eur. J. Med. Chem., (2018), 147, 227-237] or isoindolines [Grundmana, M. et al., Alzheimer's & Dementia: Translational Research & Clinical Interventions, (2019), 5, 20-26] amongst others [Berardi, F. et al., J. Med. Chem., (2004), 47, 2308-2317]. Many of these ligands present a lack of selectivity over serotoninergic receptors but mainly, there is a difficulty to reach a high selectivity over σ₁. Several σ₁-selective ligands are available, but ligands with high selectivity for σ₂ over σ₁ are relatively scarce. A significant challenge for the study of the σ₂ receptor is the paucity of highly σ₂-selective ligands.

In view of the potential therapeutic applications of agonists or antagonists of the sigma receptor, a great effort has been directed to find selective ligands. Thus, the prior art has disclosed different sigma receptor ligands, as outlined above.

Nevertheless, there is still a need to find compounds having pharmacological activity towards the sigma receptor, being both effective, selective, and/or having good “drugability” properties, i.e. good pharmaceutical properties related to administration, distribution, metabolism and excretion.

Surprisingly, it has been observed that the new compounds described in the present invention show a selective affinity for sigma receptors. These compounds are therefore particularly suitable as pharmacologically active agents in medicaments for the prophylaxis and/or treatment of disorders or diseases related to sigma receptors.

### SUMMARY OF THE INVENTION

The present invention discloses novel compounds with great affinity to sigma receptors which might be used for the treatment of sigma related disorders or diseases. In particular, the compounds of the invention can be useful for the treatment of pain and pain related disorders and/or CNS (Central Nervous System) disorders.

The invention is directed in a main aspect to a compound of Formula (I),

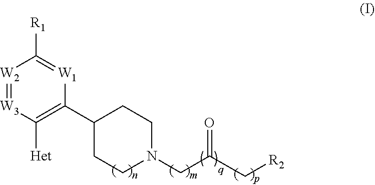

wherein R₁, R₂, Het, W₁, W₂, W₃, n, m, q, and p are as defined below in the detailed description.

A further aspect of the invention refers to the processes for preparation of compounds of formula (I).

It is also an aspect of the invention a pharmaceutical composition comprising a compound of formula (I).

Finally, it is an aspect of the invention a compound of formula (I) for use in therapy and more particularly for the treatment of pain and pain related conditions and/or CNS (Central Nervous System) disorders.

### DETAILED DESCRIPTION OF THE INVENTION

The invention is directed to a family of new compounds, which show a pharmacological activity towards the sigma receptors; thus, solving the above problem of identifying alternative or improved pain and/or CNS treatments by offering such compounds.

The applicant has found that the problem of providing a new effective and alternative solution for treating pain and pain related disorders and/or CNS (Central Nervous System) disorders can surprisingly be solved by using compounds binding to the sigma receptors.

In a first aspect, the present invention is directed to a compound of formula (I):

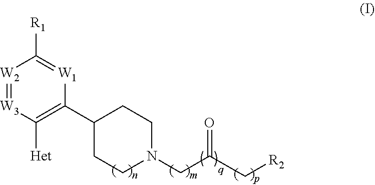

wherein:
n is selected from 1 or 2
m is selected from 1 or 2
p is selected from 0 or 1
q is selected from 0 or 1
W₁, W₂, W₃ is —CH— or —N— wherein at least one of them is —N—
R₁ is a linear or branched C₁-C₆ alkyl radical; C₁-C₆ haloalkyl; or C₃-6 cycloalkyl optionally substituted;
Het is an optionally mono or polysubstituted C₃-C₉ heterocyclyl radical having at least one heteroatom selected from the group N, O or S;
R₂ is

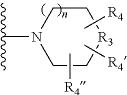

wherein
R₃ is selected from —CH₂—, —CH—(R₄), —C—(R₄)(R₄), —N—(R₄) or —O—; and R₄, R₄′ R₄″ are independently from one another selected from H, C₁₋₆-alkyl, C₁₋₆-haloalkyl, C₁₋₆ alkoxy, —CN, —NRR′, —wherein R and R′ are independently selected from hydrogen or C₁₋₆-alkyl;
or alternatively when R₄ and R₄′ are attached to the same carbon atom, they can form together a carbocyclic or heterocyclic spiro ring;
with the proviso that the following compound is excluded:
2-[4-[2-methyl-5-(3-methyl-5-isoxazolyl)-4-pyrimidinyl]-1-piperidinyl]-1-(4-morpholinyl)-ethanone

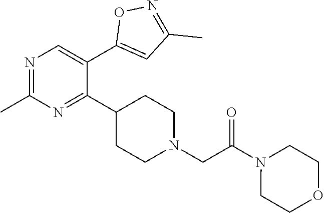 [SMILES: Cc1cc(-c2cnc(C)nc2C2CCN(CC(=O)N3CCOCC3)CC2)on1]

wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

For the sake of clarity the expression “a compound according to formula (I), wherein R₁, R₂, Het, W₁, W₂, W₃, n, m, q, and p are as defined below in the detailed description” would (just like the expression “a compound of formula (I) as defined in the claims) refer to “a compound according to formula (I)”, wherein the definitions of the respective substituents R₁ etc. (also from the cited claims) are applied.

For clarity purposes, all groups and definitions described in the present description and referring to compounds of formula (I), also apply to all intermediates of synthesis.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

“C₁₋₆ alkyl”, as referred to in the present invention, are saturated aliphatic radicals. They may be unbranched (linear) or branched and are optionally substituted. C₁₋₆-alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl, 1-methylpentyl. The most preferred alkyl radicals are C₁₋₄ alkyl, such as methyl, ethyl, propyl, n-propyl, isopropyl, butyl, n-butyl, tert-butyl, isobutyl, sec-butyl, 1-methylpropyl, 2-methylpropyl or 1,1-dimethylethyl. Alkyl radicals, as defined in the present invention, are optionally mono- or polysubstituted by substitutents independently selected from a halogen, branched or unbranched C₁₋₆-alkoxy, branched or unbranched C₁₋₆-alkyl, C₁₋₆-haloalcoxy, C₁₋₆-haloalkyl, trihaloalkyl or a hydroxyl group.

“C₁₋₆ alkoxy” as referered to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy. “C₃₋₆ Cycloalkyl” as referred to in the present invention, is understood as meaning saturated and unsaturated (but not aromatic), cyclic hydrocarbons having from 3 to 6 carbon atoms which can optionally be unsubstituted, mono- or polysubstituted. Examples for cycloalkyl radical preferably include but are not restricted to cyclopropyl, cyclobutyl, cyclopentyl, cyclohexyl. The most preferred cycloalkyl radical is cyclopropyl. Cycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A cycloalkylalkyl group/radical C_{1-6,} as defined in the present invention, comprises a branched or unbranched, optionally at least mono-substituted alkyl chain of 1 to 6 atoms which is bonded to a cycloalklyl group, as defined above. The cycloalkylalkyl radical is bonded to the molecule through the alkyl chain. A preferred cycloalkylalkyl group/radical is a cyclopropylmethyl group or a cyclopentylpropyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A heterocyclyl radical (Het) or group (also called heterocyclyl hereinafter) is understood as meaning 3 to 9 membered mono or fused polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 3 to 9 membered mono or fused polycyclic heterocyclic ring system of one or more rings of which at least one aromatic ring contains one or more heteroatoms from the group consisting of nitrogen, oxygen and/or sulfur in the ring; preferably it is a 3 to 9 membered mono or fused polycyclic aromatic heterocyclic ring system of one or two rings of which at least one aromatic ring contains one or more heteroatoms selected from the group consisting of nitrogen, oxygen and/or sulfur in the ring; more preferably it is selected from furan, benzofuran, thiophene, benzothiophene, pyrrole, pyridine, pyrimidine, pyrazine, benzothiazole, indole, benzotriazole, thiazole, imidazole, pyrazole, oxazole, oxadiazoleand benzimidazole;
the non-aromatic heterocyclyl is a 3 to 9 membered mono or fused polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably it is a 3 to 9 membered mono or fused polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably it is selected from azetidine, oxetane, tetrahydrofuran, oxazepane, pyrrolidine, piperidine, piperazine, tetrahydropyran, morpholine, indoline, oxopyrrolidine, especially is piperazine, morpholine, tetrahydropyran, piperidine, oxopyrrolidine and pyrrolidine.

Preferably, in the context of this invention heterocyclyl is defined as a 3 to 9 membered mono or fused polycyclic ring system of one or more saturated or unsaturated rings of which at least one ring contains one or more heteroatoms selected from the group consisting of nitrogen, oxygen and/or sulfur in the ring. Preferably it is a 3 to 9 membered mono or fused polycyclic heterocyclic ring system of one or two saturated or unsaturated rings of which at least one ring contains one or more heteroatoms selected from the group consisting of nitrogen, oxygen, and sulfur in the ring. More preferably, it is a 3 to 6 membered mono or bicyclic heterocyclyl ring system containing one nitrogen atom and optionally a second heteroatom selected from nitrogen and oxygen. In another preferred embodiment of the invention, said heterocyclyl is a substituted mono or bicyclic heterocyclyl ring system.

Preferred examples of heterocyclyls include azetidine, azepane, oxetane, tetrahydrofuran, oxazepane, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, benzo-1,2,5-thiadiazole, indole, benzotriazole, benzoxazole oxopyrrolidine, benzodioxolane, benzodioxane, 2,7-diazaspiro[3.5]nonane, 2,7-diazaspiro[4.4]nonane, octahydropyrrolo[3,4-c]pyrrole, especially is pyridine, piperazine, pyrazine, indazole, benzodioxane, thiazole, benzothiazole, morpholine, tetrahydropyran, pyrazole, imidazole, piperidine, thiophene, indole, benzimidazole, pyrrolo[2,3-b]pyridine, benzoxazole, oxopyrrolidine, pyrimidine, oxazepane, pyrrolidine, azetidine, azepane, oxetane, tetrahydrofuran, and 2,7-diazaspiro[3.5]nonane.

An N-containing heterocyclyl is a heterocyclic ring system of one or more saturated or unsaturated rings of which at least one ring contains a nitrogen and optionally one or more further heteroatoms selected from the group consisting of nitrogen, oxygen and/or sulfur in the ring; preferably is a heterocyclic ring system of one or two saturated or unsaturated rings of which at least one ring contains a nitrogen and optionally one or more further heteroatoms selected from the group consisting of nitrogen, oxygen and/or sulfur in the ring, more preferably is selected from azetidine, azepane, oxazepam, pyrrolidine, imidazole, oxadiazole, tetrazole, azetidine, pyridine, pyrimidine, piperidine, piperazine, benzimidazole, indazole, benzothiazole, benzodiazole, morpholine, indoline, triazole, isoxazole, pyrazole, pyrrole, pyrazine, pyrrolo[2,3-b]pyridine, tetrahydrothienopyridine, benzo-1,2,5-thiadiazole, indole, benzotriazole, benzoxazole oxopyrrolidine, thiazole, 2,7-diazaspiro[3.5]nonane, 2,7-diazaspiro[4.4]nonane or octahydropyrrolo[3,4-c]pyrrole.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

“Heterocycloalkyl” as referred to in the present invention, are understood as meaning saturated and unsaturated (but not aromatic), generally 5 or 6 membered cyclic hydrocarbons which can optionally be unsubstituted, mono- or polysubstituted and which have at least one heteroatom in their structure selected from N, O or S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalkoxy, C₁₋₆-haloalkyl, trihaloalkyl or a hydroxyl group. More preferably heterocycloalkyl in the context of the present invention are 5 or 6-membered ring systems optionally at least monosubstituted.

A heterocycloalkylalkyl group/radical C₁₋₆, as defined in the present invention, comprises a linear or branched, optionally at least mono-substituted alkyl chain of 1 to 6 atoms which is bonded to a cycloalklyl group, as defined above. The heterocycloalkylalkyl radical is bonded to the molecule through the alkyl chain. A preferred heterocycloalkylalkyl group/radical is a piperidinethyl group or a piperazinylmethyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Aryl” as referred to in the present invention, is understood as meaning ring systems with at least one aromatic ring but without heteroatoms even in only one of the rings. These aryl radicals may optionally be mono-or polysubstituted by substitutents independently selected from a halogen atom, —CN, branched or unbranched C₁₋₆-alkyl, branched or unbranched C₁₋₆-alkoxy, C₁₋₆-haloalcoxy, C₁₋₆-haloalkyl, a heterocyclyl group and a hydroxyl group. Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention is a 6-membered ring system optionally at least mono or polysubstituted.

An arylalkyl radical C_{1-6,} as defined in the present invention, comprises an unbranched or branched, optionally at least mono-substituted alkyl chain of 1 to 6 carbon atoms which is bonded to an aryl group, as defined above. The arylalkyl radical is bonded to the molecule through the alkyl chain. A preferred arylalkyl radical is a benzyl group or a phenetyl group, wherein the alkyl chain is optionally branched or substituted. Preferred substituents for arylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Heteroaryl” as referred to in the present invention, is understood as meaning heterocyclic ring systems which have at least one aromatic ring and contain one or more heteroatoms from the group consisting of N, O or S and may optionally be mono-or polysubstituted by substituents independently selected from a halogen atom, branched or unbranched C₁₋₆-alkyl, branched or unbranched C₁₋₆-alkoxy, C₁₋₆-haloalkoxy, C₁₋₆-haloalkyl trihaloalkyl or a hydroxyl group. Preferred examples of heteroaryls include but are not restricted to furan, benzofuran, pyrrole, pyridine, pyrimidine, pyridazine, pyrazine, quinoline, isoquinoline, phthalazine, triazole, pyrazole, isoxazole, indole, benzotriazole, benzodioxolane, benzodioxane, benzimidazole, carbazole and quinazoline. More preferably heteroaryl in the context of the present invention are 5 or 6-membered ring systems optionally at least monosubstituted.

Heteroarylalkyl group/radical C₁₋₆ as defined in the present invention, comprises a linear or branched, optionally at least mono-substituted alkyl chain of 1 to 6 carbon atoms which is bonded to an heteroaryl group, as defined above. The heteroarylalkyl radical is bonded to the molecule through the alkyl chain. A preferred heteroarylalkyl radical is a piridinylmethyl group, wherein the alkyl chain is optionally branched or substituted. Preferred substituents for heteroarylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

The term “condensed” according to the present invention means that a ring or ring-system is attached to another ring or ring-system, whereby the terms “annulated” or “annelated” are also used by those skilled in the art to designate this kind of attachment.

The term “ring system” according to the present invention refers to a system consisting of at least one ring of connected atoms but including also systems in which two or more rings of connected atoms are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

A leaving group (LG) is a group that in a heterolytic bond cleavage keeps the electron pair of the bond. Suitable leaving groups are well known in the art and include Cl, Br, I and —O—SO₂R¹⁴, wherein R¹⁴ is F, C₁₋₄-alkyl, C₁₋₄-haloalkyl, or optionally substituted phenyl. The preferred leaving groups are Cl, Br, I, tosylate, mesylate, triflate, nonaflate and fluorosulphonate.

“Protecting group” is a group that is chemically introduced into a molecule to avoid that a certain functional group from that molecule undesirably reacts in a subsequent reaction. Protecting groups are used, among others, to obtain chemoselectivity in chemical reactions. The preferred protecting group in the context of the invention are Boc (tert-butoxycarbonyl) or Teoc (2-(trimethylsilyl)ethoxycarbonyl).

The term “salt” is to be understood as meaning any form of the active compound according to the invention in which this assumes an ionic form or is charged and is coupled with a counter-ion (a cation or anion). The definition particularly includes physiologically acceptable salts, this term must be understood as equivalent to “pharmaceutically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid. In addition, the pharmaceutically acceptable salts may be formed with a physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals.

The compounds of the invention may be present in crystalline form or in amorphous form.

Any compound that is a solvate of a compound according to formula (I) defined above is understood to be also covered by the scope of the invention. Methods of solvation are generally known within the art. Suitable solvates are pharmaceutically acceptable solvates. The term “solvate” is to be understood as meaning any form of the active compound according to the invention in which this compound has attached to it via non-covalent binding another molecule (most likely a polar solvent) especially including hydrates and alcoholates, like methanolate or ethanolate.

The term “co-crystal” is to be understood as a crystalline material comprising a specific active compound with at least one additional component, usually a co-crystal former, and of which at least two of the constituents are held together by weak interactions. Weak interaction is being defined as an interaction which is neither ionic nor covalent and includes for example: hydrogen bonds, van der Waals forces, and x-x interactions.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of general formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is an N-oxide of a compound according to the invention like a compound according to formula (I) defined above is understood to be also covered by the scope of the invention.

The compounds of formula (I) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

Unless otherwise defined, all the groups above mentioned that can be substituted or unsubstituted may be substituted at one or more available positions by one or more suitable groups such as a halogen, preferably Cl or F; OR′, ═O, SR′, SOR′, SO₂R′, OSO₂R′, OSO₃R′, NO₂, NHR′, NR′R″, ═N—R′, N(R′)COR′, N(COR′)₂, N(R′)SO₂R′, N(R′)C(═NR′)N(R′)R′, N₃, CN, halogen, COR′, COOR′, OCOR′, OCOOR′, OCONHR′, OCONR′R″, CONHR′, CONR′R″, CON(R′)OR′, CON(R′)SO₂R′, PO(OR′)₂, PO(OR′)R′, PO(OR′)(N(R′)R′), C₁₋₆ alkyl, C₃₋₁₀ cycloalkyl, C₂₋₆ alkenyl, C₂₋₆ alkynyl, aryl, and heterocyclic group, wherein each of the R′ and R″ groups is independently selected from the group consisting of hydrogen, C₁₋₆ alkyl, C₃₋₁₀ cycloalkyl, C₂₋₆ alkenyl, C₂₋₆ alkynyl, aryl and heterocyclic group. Where such groups are themselves substituted, the substituents may be chosen from the foregoing list.

In a particular and preferred embodiment of the invention, R₁ is selected from: a C₁-C₆ alkyl radical, branched or unbranched C₃₋₆ cycloalkyl or C₁₋₆-haloalkyl. In an even more preferably embodiment R₁ is selected from: ethyl, propyl, isopropyl cyclopropyl; or trifluoromethyl.

In another particular and preferred embodiment, the heterocyclic ring radical, Het is selected from an optionally substituted 5-membered aromatic ring which contains two heteroatoms N and O, even more preferably the Het radical is an isoxazole ring optionally substituted with C₁₋₆ alkyl, preferably a methyl.

In still another preferred embodiment Het is selected from an optionally substituted 5 or 6-membered aromatic ring which contains one or two N atoms, even more preferably the Het radical is a pyrimidine ring, pyridine ring or imidazole ring all optionally monosubstituted with a C₁₋₆ alkyl, preferably a methyl.

In another particular and preferred embodiment R₂ is a group selected from:

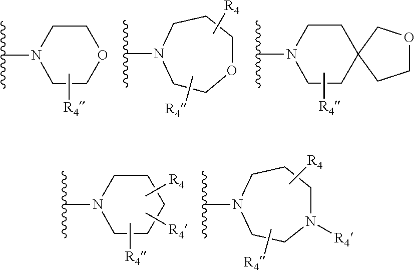

wherein R₄, R₄′ R₄″ independently from one another is selected from: H, C₁₋₄-alkyl, C₁₋₄-haloalkyl, C₁₋₄ alkoxy, —CN, —NRR′, —wherein R and R′ are independently selected from hydrogen or C₁₋₆-alkyl, even more preferably R₄, R₄′ R₄″ independently from one another is selected from: H, metal and fluor.

In yet another preferred embodiment, Ws is —CH— and W₁ and W₂ are —N— or W₁ is —N— and W₂ and W₃ are —CH—.

A further particular and preferred embodiment of the invention comprises a compound of formula (I):

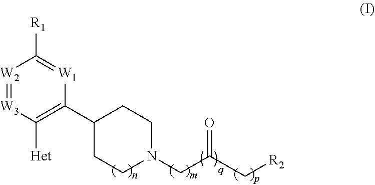

wherein:
n is selected from 1 or 2
m is selected from 1 or 2
p is selected from 0 or 1
q is selected from 0 or 1
W₁, W₂, W₃ is —CH— or —N— wherein at least one of them is —N—
R₁ is selected from: ethyl, propyl, isopropyl, cyclopropyl; or trifluoromethyl;
Het is selected from an optionally monosubstituted 5 or 6-membered aromatic ring which contains two heteroatoms independently selected from N and O;
R₂ is selected from:

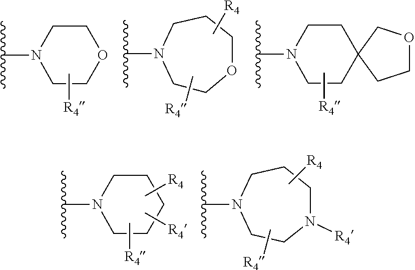

with the proviso that the following compound is excluded:
2-[4-[2-methyl-5-(3-methyl-5-isoxazolyl)-4-pyrimidinyl]-1-piperidinyl]-1-(4-morpholinyl)-ethanone

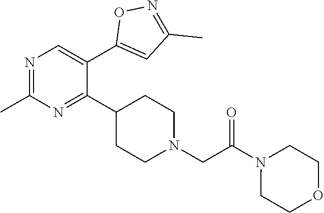 [SMILES: Cc1cc(-c2cnc(C)nc2C2CCN(CC(=O)N3CCOCC3)CC2)on1]

wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

The preferred compounds of the invention are selected from:
[1] 2-(4-(2-Cyclopropyl-5-(3-methylisoxazol-5-yl)pyrimidin-4-yl)piperidin-1-yl)-1-morpholinoethanone;
[2] 2-(4-(2-Isopropyl-5-(3-methylisoxazol-5-yl)pyrimidin-4-yl)piperidin-1-yl)-1-morpholinoethanone;
[3] 2-(4-(2-Ethyl-5-(3-methylisoxazol-5-yl)pyrimidin-4-yl)piperidin-1-yl)-1-(1,4-oxazepan-4-yl)ethanone;
[4] 2-(4-(2-Ethyl-5-(3-methylisoxazol-5-yl)pyrimidin-4-yl)piperidin-1-yl)-1-(2-oxa-8-azaspiro[4.5]decan-8-yl)ethanone;
[5] 2-(4-(2-Ethyl-5-(3-methylisoxazol-5-yl)pyrimidin-4-yl)piperidin-1-yl)-1-morpholinoethanone;
[6] 1-(4,4-Difluoropiperidin-1-yl)-2-(4-(2-ethyl-5-(3-methylisoxazol-5-yl)pyrimidin-4-yl)piperidin-1-yl)ethanone;
[7] 2-(4-(2-Ethyl-5-(3-methylisoxazol-5-yl)pyrimidin-4-yl)piperidin-1-yl)-1-(4-methyl-1,4-diazepan-1-yl)ethanone;
[8] 2-(4-(5-(3-Methylisoxazol-5-yl)-2-(trifluoromethyl)pyrimidin-4-yl)piperidin-1-yl)-1-morpholinoethanone;
[9] 3-(4-(2-Ethyl-5-(3-methylisoxazol-5-yl)pyrimidin-4-yl)piperidin-1-yl)-1-morpholinopropan-1-one;
[10] 4-(2-(4-(2-Ethyl-5-(3-methylisoxazol-5-yl)pyrimidin-4-yl)piperidin-1-yl)ethyl)morpholine;
[11] 2-(4-(2-Ethyl-5-(3-methylisoxazol-5-yl)pyrimidin-4-yl)piperidin-1-yl)-1-(piperidin-1-yl)ethan-1-one;
[12] 2-(4-(5-(3-Methylisoxazol-5-yl)-2-propylpyrimidin-4-yl)piperidin-1-yl)-1-morpholinoethan-1-one;
[13] 2-(4-(2-Ethyl-5-(pyridin-2-yl)pyrimidin-4-yl)piperidin-1-yl)-1-morpholinoethan-1-one;
[14] 2-(4-(2-Ethyl-5-(6-methylpyridin-2-yl)pyrimidin-4-yl)piperidin-1-yl)-1-morpholinoethan-1-one;
[15] 2-(4-(2-Ethyl-5-(pyridin-4-yl)pyrimidin-4-yl)piperidin-1-yl)-1-morpholinoethan-1-one;
[16] 2-(4-(2-Ethyl-5-(1-methyl-1H-imidazol-2-yl)pyrimidin-4-yl)piperidin-1-yl)-1-morpholinoethan-1-one;
[17] (R)-2-(4-(2-Ethyl-5-(3-methylisoxazol-5-yl)pyrimidin-4-yl)azepan-1-yl)-1-morpholinoethan-1-one;
[18] (S)-2-(4-(2-Ethyl-5-(3-methylisoxazol-5-yl)pyrimidin-4-yl)azepan-1-yl)-1-morpholinoethan-1-one; and
[19] 2-(4-(6-Methyl-3-(2-methylpyrimidin-4-yl)pyridin-2-yl)piperidin-1-yl)-1-morpholinoethan-1-one.

In another aspect, the invention refers to the processes for obtaining the compounds of general formula (I). Several procedures have been developed for obtaining all the compounds of the invention, and the procedures will be explained below in methods A and B.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

### Method A

Method A represents a first process for synthesizing compounds according to general formula (Ia). Method A allows the preparation of compounds of general formula (Ia) that is compounds of general formula (Ia) where W₂, W₃ is N and W₁ is C.

Thus, a process is described for the preparation of a compound of general formula (Ia):

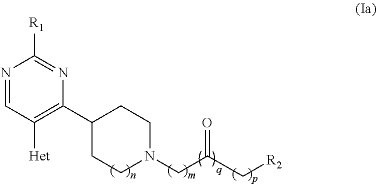

comprising the reaction of a compound of formula (VIII):

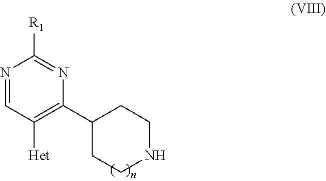

with a compound of formula (IX):

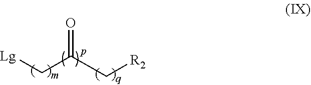

wherein R₁, R₂, Het, n, m, p and q are as defined along the detailed description and the claims and LG (leaving group) is a suitable leaving group as defined along the detailed description.

The compound of formula (I) can be obtained by alkylation of a compound of formula (VIII) with a compound of formula (IX) in the presence of a suitable base preferably triethylamine in a suitable solvent, preferably MeCN, at a suitable temperature, preferably at room temperature.

Scheme 1 below summarizes the synthetic route that leads to compounds of formula (VIII) and the alkylation step of method A.

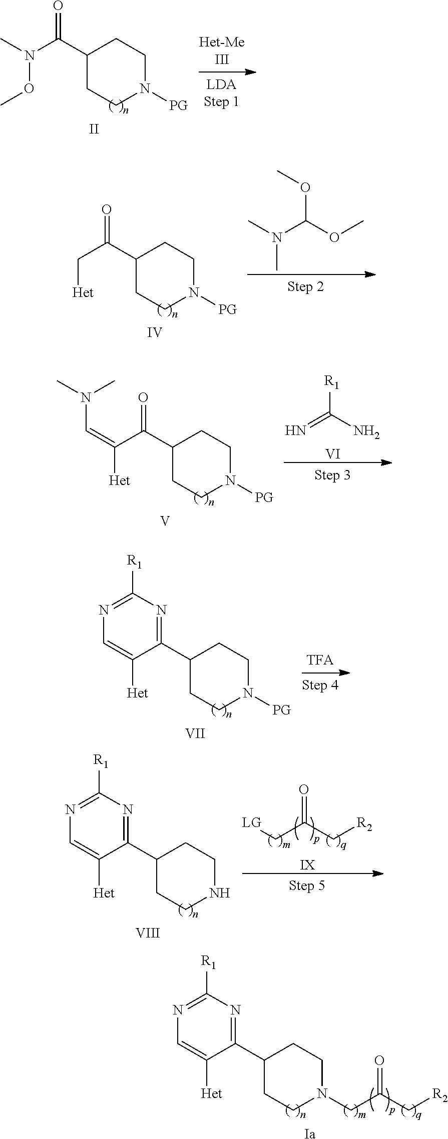

wherein R₁, R₂, Het, n, m, p, q, LG and PG (protecting group) have the meanings as defined along the detailed description and/or the claims.

In a preferred embodiment the process can be carried out as described below:

Step 1: A compound of formula (IV) can be prepared from a compound of formula (II) by treatment with a reagent of formula (III) in the presence of an organometallic reagent such as an organolithium reagent, preferably LDA, in a suitable solvent such as THF, at a suitable temperature, preferably at −78° C.

Step 2: A compound of formula (V)can be obtained by reaction of a compound of formula (IV) with N,N-dimethylformamide dimethyl acetal in a suitable solvent such as toluene, at a suitable temperature, preferably at 110° C.

Step 3: A compound of formula (VII) can be prepared by treatment of a compound of formula (V) with a suitable amidine of formula (VI) in the presence of a base, such as potassium carbonate, in a suitable solvent such as EtOH, at a suitable temperature, preferably at 80° C.

Step 4: A compound of formula (VIII) can be prepared by removal of the amine protecting group of a compound of formula (VII) by treatment with a suitable agent such as TFA in a suitable solvent, such as dichloromethane, at a suitable temperature, preferably at room temperature.

Step 5: A compound of formula (I) can be obtained by alkylation of a compound of formula (VIII) with a compound of formula (IX) in the presence of a suitable base such as triethylamine in a suitable solvent, such as MeCN, at a suitable temperature, preferably at room temperature.

### Method B

Method B represents a second process for synthesizing compounds according to general formula (I).

Therefore, a process is described for the preparation of a compound of formula (I):

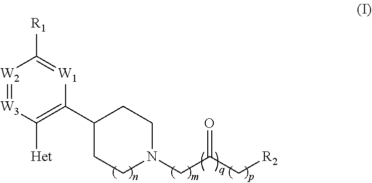

comprising the reaction of a compound of formula (XIX):

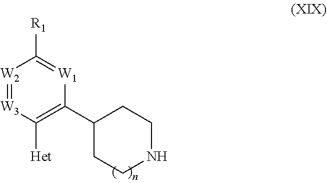

with a compound of formula (IX):

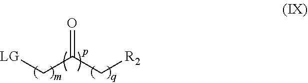

wherein R₁, R₂, W₁, W₂, W₃, Het, n, m, p, q and LG are as defined in the claims and/or along the description.

Scheme 2 below summarizes the synthetic route that leads to compounds of formula (XIX) and the alkylation step of method B.

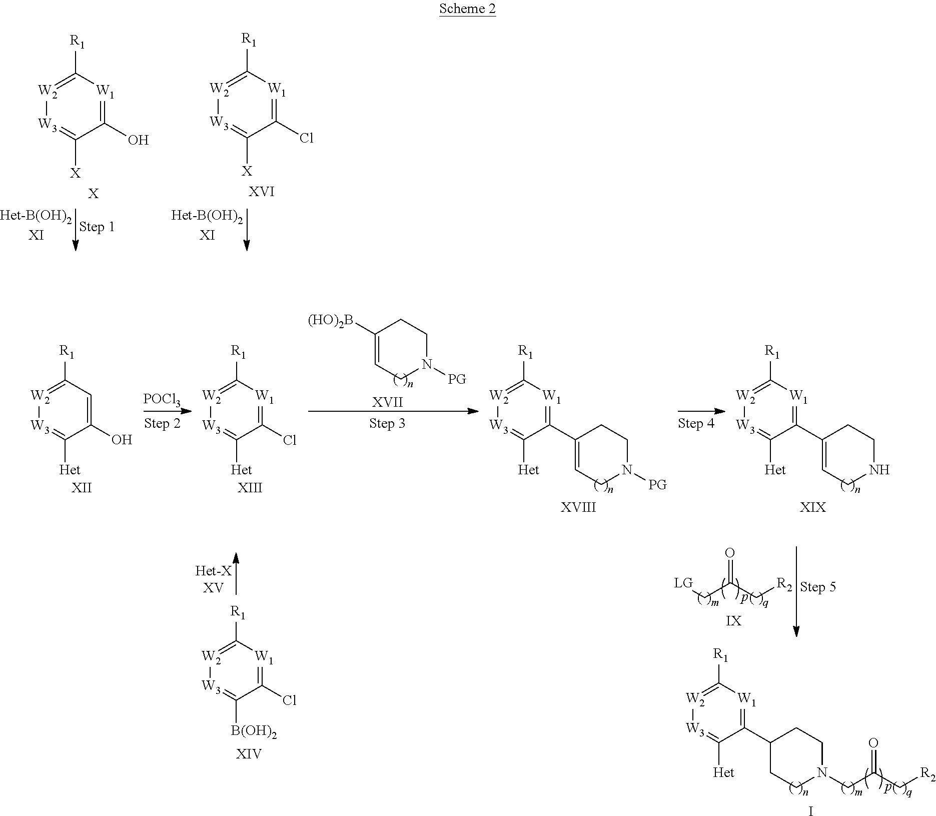

wherein R₁, R₂, W₁, W₂, W₃, Het, n, m, p, and q have the meanings as defined in claim 1, PG is a protecting group, R is H or alkyl and X is a suitable halogen atom.

The process can be carried out as described below:

Step 1: A compound of formula (XII) can be prepared by treating a compound of formula (X) with a boronic acid of formula (XI) (or alternatively with the corresponding boronic ester) in the presence of a Pd catalyst, such as Pd(PPh₃)₄ and a suitable base, such as Na₂CO₃ or K₂CO₃, in a suitable solvent such as N,N-dimethylformamide or a mixture of dioxane and water, at a suitable temperature, preferably reflux temperature, optionally under microwave irradiation.

Step 2: A compound of formula (XIII) can be obtained from a compound of formula (XII) by treatment with a chlorinating agent such as phosphorous oxychloride, at a suitable temperature, preferably reflux temperature.

Alternatively, a compound of formula (XIII) can be prepared from a boronic acid (or alternatively with the corresponding boronic ester) of formula (XIV) by reaction with a compound of formula (XV) under similar conditions described in step 1. Following similar Suzuki conditions, compounds of formula (XIII) can also be obtained from compounds of formula (XVI) by treatment with boronic acids (or alternatively with the corresponding boronic ester) of formula (XI).

Step 3: A compound of formula (XVIII) can be prepared by treating a compound of formula (XIII) with a compound of formula (XVII) (or alternatively with the corresponding boronic ester) under suitable Suzuki conditions described in step 1. When n=2, (XVII) can be a tetrahydroazepine as drawn in Scheme 2, with the double bond in 4,5-positions or, alternatively it can be the isomer with the double bond in 5,6 positions, (XVII′), to give the corresponding isomer (XVIII′).

Step 4: A compound of formula (XIX) can be prepared by reduction of the double bond and protecting group removal of a compound of formula (XVIII) or (XVIII′) under hydrogen transfer conditions in the presence of ammonium formate or under hydrogen pressure with suitable Pd catalysis, in a suitable solvent, such as methanol, at a suitable temperature, preferably under reflux for hydrogen transfer conditions, or at room temperature under hydrogen pressure. Depending on the nature of the PG, an additional step may be necessary for its removal.

Step 5: A compound of formula (I) can be obtained by alkylation of a compound of formula (XIX) with a compound of formula (IX) in the presence of a suitable base such as triethylamine in a suitable solvent, such as MeCN, at a suitable temperature, preferably at room temperature.

In both Scheme 1 and 2, it may be possible to avoid the use of the amine protecting groups PG and carry out the reaction sequence with the final substituent (CH₂)ₘ(CO)ₚ(CH₂)_{q}R₂.

In some of the processes described above it may be necessary to protect the reactive or labile groups present with suitable protecting groups, such as for example acetyl, allyl, Alloc (allyloxycarbonyl), Boc (tert-butoxycarbonyl), or benzyl for the protection of amino groups, and common silyl protecting groups for the protection of the hydroxyl group. The procedures for the introduction and removal of these protecting groups are well known in the art and can be found thoroughly described in the literature.

In addition, a compound of formula (I) can be obtained in enantiopure form by resolution of a mixture of enantiomers or diastereomers of formula (I) either by chiral preparative HPLC or by crystallization of a diastereomeric salt or co-crystal. Alternatively, the resolution step can be carried out at a previous stage, using any suitable intermediate.

The compounds of formula (II), (III), (VI), (IX), (X), (XI), (XIV), (XV), (XVI) and (XVII) used in the methods disclosed above are commercially available or can be synthesized following common procedures described in the literature and exemplified in the synthesis of some intermediates.

Turning to another aspect, the invention also relates to the therapeutic use of the compounds of general formula (I). As mentioned above, compounds of general formula (I) show a strong affinity to sigma receptors, especially to sigma-1 receptors and can behave as agonists, antagonists, inverse agonists, partial antagonists or partial agonists thereof. Therefore, compounds of general formula (I) are useful as medicaments.

They are suitable for the treatment and/or prophylaxis of diseases and/or disorders mediated by sigma receptors and preferably by sigma-1 receptors. In this sense, compounds of formula (I) are suitable for the treatment and/or prophylaxis of pain, especially neuropathic pain, inflammatory pain, and chronic pain or other pain conditions involving allodynia and/or hyperalgesia, or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

The compounds of general formula (I) are especially suited for the treatment of pain, especially neuropathic pain, inflammatory pain or other pain conditions involving allodynia and/or hyperalgesia or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

PAIN is defined by the International Association for the Study of Pain (IASP) as “an unpleasant sensory and emotional experience associated with actual or potential tissue damage, or described in terms of such damage (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment the compounds of the invention are used for the treatment and/or prophylaxis of neuropathic pain and more specifically for the treatment and/or prophylaxis of hyperpathia.

A related aspect of the invention refers to the use of compounds of general formula (I) for the manufacture of a medicament for the treatment and/or prophylaxis of disorders and diseases mediated by sigma receceptors and more preferably by sigma-1 receptors, as explained before.

Another related aspect of the invention refers to a method for the treatment and/or prophylaxis of disorders and diseases mediated by sigma receceptors and more preferably by sigma-1 receptors, as explained before comprising the administration of a therapeutically effective amount of a compound of general formula (I) to a subject in need thereof.

Another aspect of the invention is a pharmaceutical composition, which comprises at least a compound of general formula (I) or a pharmaceutically acceptable salt, isomer, co-crystal, prodrug or solvate thereof, and at least a pharmaceutically acceptable carrier, additive, adjuvant or vehicle.

The pharmaceutical composition of the invention can be formulated as a medicament in different pharmaceutical forms comprising at least a compound binding to the sigma receptor and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathekal, rectal, transdermal, transmucosal or nasal administration.

The composition of the invention can be formulated for oral administration in any form preferably selected from the group consisting of tablets, drageés, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the apropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

In the next examples the preparation of the compounds according to the invention is disclosed.

The following abbreviations are used in the intermediates and examples:
Aq: aqueous
Chx: cyclohexane
Et₂O: diethyl ether
EtOAc: ethyl acetate
EtOH: ethanol
EX: example
h: hours
HPLC: High Performance Liquid Chromatography
MeCN: acetonitrile
MeOH: methanol
MS: mass spectrometry
Min.: minutes
Quant: quantitative
Rt.: retention time
rt: room temperature
Sat: saturated
Sol: solution
TFA: trifluoroacetic acid
TFE: trifluoroethanol
THF: tetrahydrofuran
Wt: weight

The following methods were used to determine the HPLC-MS spectra:

### Method A

### “Agilent”

Column ZORBAX Extend-C18 RRHD 2.1×50 mm, 1.8 μm, temperature 35° C.; flow rate 0.61 mL/min; A: NH₄HCO₃ 10 mM, B: MeCN; gradient 0.3 min 98% A, 98% A to 100% B in 2.65 min; isocratic 2.05 min 100% B.

### Method B

### “Acquity”

Column ZORBAX Extend-C18 RRHD 2.1×50 mm, 1.8 μm, temperature 35° C.; flow rate 0.61 mL/min; A: NH₄HCO₃ 10 mM, B: MeCN, C: MeOH+0.1% formic acid; gradient 0.3 min 98% A, 98% A to 0:95:5 A:B:C in 2.7 min; 0:95:5 A:B:C to 100% B in 0.1 min; isocratic 2 min 100% B.

### Synthesis of Intermediates

Intermediate 1. 2-Chloro-1-morpholinoethanone.

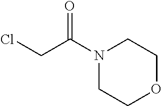

To a solution of morpholine (233 μL, 2.7 mol) and triethylamine (1.32 mL, 9.45 mmol) in CH₂Cl₂ (30 mL) 2-chloroacetyl chloride (247 μL, 3.11 mmol) in CH₂Cl2 (5 mL) was added at 0° C. The reaction mixture was stirred for 1 h at 0° C.and then at rt for 3 h. The reaction was quenched with sat. aq. NaHCO₃ solution and the product extracted with CH₂Cl₂. The combined organic layers were dried over Na₂SO₄, filtered and concentrated to dryness to give the title compound (366 mg, 83% yield) as gold oil.

HPLC Rt (Method A): 0.81 min; ESI+−MS m/z: 164 (M+H)⁺.

¹H NMR (CDCl₃) δ: 4.06 (s, 2H), 3.75-3.68 (m, 4H), 3.66-3.60 (m, 2H), 3.57-3.50 (m, 2H).

This method was used for the preparation of Intermediates 2-5 using suitable starting materials:

| INT | STRUCTURE | CHEMICAL NAME |
|---|---|---|
| 2 | 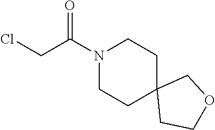 [SMILES: O=C(CCl)N1CCC2(CCOC2)CC1] | 2-Chloro-1-(2-oxa-8- azaspiro[4.5]decan- 8-yl)ethanone |
| 3 | 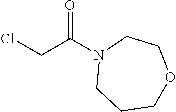 [SMILES: O=C(CCl)N1CCCOCC1] | 2-Chloro-1-(1,4- oxazepan-4- yl)ethenone |
| 4¹ | 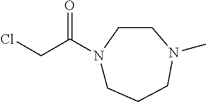 | 2-Chloro-1-(4-methyl- 1,4-diazepan-1- yl)ethenone |
| 5 | 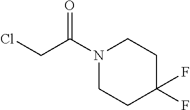 [SMILES: O=C(CCl)N1CCC(F)(F)CC1] | 2-Chloro-1-(4,4- difluoropiperidin-1- yl)ethenone |

¹This intermediate was used directly in solution without aqueous workup.

Intermediate 6. 3-Chloro-1-morpholinopropan-1-one.

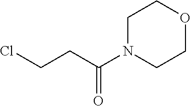 [SMILES: O=C(CCCl)N1CCOCC1]

To a solution of morpholine (248 μL, 2.87 mmol) in MeCN (10 mL) potassium carbonate (793 mg, 5.75 mmol) was added and the reaction cooled down to 0° C. At this temperature 3-chloropropanoyl chloride (301 μL, 3.16 mmol) was added and the reaction stirred for 30 min. at 0° C. The reaction was quenched with sat. aq. NaHCO₃ solution and the product extracted with EtOAc. The combined organic layers were dried over Na₂SO₄, filtered and concentrated to dryness to give the title compound (quant.).

¹H NMR (CDCl₃) δ: 3.84 (t, J=7.0 Hz, 2H), 3.71-3.66 (m, 4H), 3.66-3.60 (m, 2H), 3.48 (t, J=4.8 Hz, 2H), 2.79 (t, J=7.0 Hz, 2H).

### Synthesis of Examples

### Example 1. 2-(4-(2-Cyclopropyl-5-(3-methylisoxazol-5-yl)pyrimidin-4-yl)piperidin-1-yl)-1-morpholinoethanone.

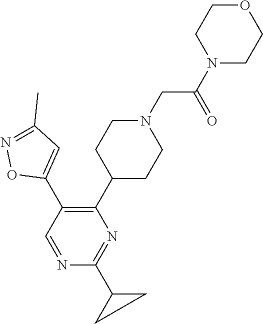 [SMILES: Cc1cc(-c2cnc(C3CC3)nc2C2CCN(CC(=O)N3CCOCC3)CC2)on1]

Step 1. tert-Butyl 4-(2-(3-methylisoxazol-5-yl)acetyl)piperidine-1-carboxylate.

A schlenk flask was charged with 3,5-dimethylisoxazole (1.44 mL, 14.7 mmol) and THF (30 mL) under argon and the solution cooled down to −78° C. Lithium diisopropylamide solution (2 M in THF, 8.81 mL, 17.6 mmol) was added and the reaction was stirred for 1 h at −78° C. before tert-butyl 4-(methoxy(methyl)carbamoyl)piperidine-1-carboxylate (2 g, 7.34 mmol) was added. The reaction was stirred for 2 h at −78° C.and then at rt overnight. The reaction was quenched with brine, the solution extracted with EtOAc and the combined organic layers were dried over Na2SO₄, filtered and concentrated to dryness. The residue was purified by flash chromatography (silica gel, Chx/EtOAc) to give the title compound (1.09 g, 48% yield).

HPLC Rt (Method A): 1.89 min; ESI+−MS m/z: 309.2 (M+H)⁺.

Step 2. (Z)-tert-Butyl 4-(3-(dimethylamino)-2-(3-methylisoxazol-5-yl)acryloyl)piperidine-1-carboxylate.

To a solution of the compound obtained in step 1 (264 mg, 0.86 mmol) in toluene (15 mL), 1,1-dimethoxy-N,N-dimethylmethanamine (170 μL, 1.28 mmol) was added. The reaction mixture was heated at reflux overnight. All volatiles were removed under reduced pressure to give the title compound (310 mg, quant.).

HPLC Rt (Method B): 1.79 min; ESI+−MS m/z: 364.3 (M+H)⁺.

Step 3. tert-Butyl 4-(2-cyclopropyl-5-(3-methylisoxazol-5-yl)pyrimidin-4-yl)piperidine-1-carboxylate.

To the compound obtained in step 2 (90 mg, 0.21 mmol) in EtOH (7 mL), cyclopropanecarboximidamide hydrochloride (27.6 mg, 0.23 mmol) followed by potassium carbonate (106 mg, 0.77 mmol) were added under argon. The reaction mixtue was heated under reflux overnight. After cooling back to rt, the solvent was removed under reduced pressure and the residue was partitioned between water and EtOAc. The aqueous layer was extracted with EtOAc and the combined organic layers were dried over Na₂SO₄, filtered and concentrated to dryness to give the title compound (74 mg, 91% yield).

HPLC Rt (Method A): 2.43 min; ESI+−MS m/z: 385.2 (M+H)⁺.

Step 4. 5-(2-Cyclopropyl-4-(piperidin-4-yl)pyrimidin-5-yl)-3-methylisoxazole 2,2,2-trifluoroacetate.

The compound obtained in Step 3 (72 mg, 0.18 mmol) was dissolved in CH₂Cl₂ (7 mL) and the reaction solution cooled to 0° C. At this temperature TFA (72 μL, 0.91 mmol) was added and the resulting solution was slowly allowed to reach rt and stirred overnight. The volatiles were removed under reduced pressure and the solid washed with Et₂O to give the title compound (quant.) as light brown solid.

HPLC Rt (Method A): 1.46 min; ESI+−MS m/z: 285.2 (M+H)⁺.

### Step 5. Title Compound.

To a solution of the compound obtained in Step 4 (55 mg, 0.09 mmol) in MeCN (3 mL) and triethylamine (64 μL, 0.46 mmol) intermediate 1 (22.4 mg, 0.14 mmol) was added at 0° C. The reaction mixture was stirred at rt overnight. All volatiles were removed under reduced pressure and the residue partitioned between aqueous NaOH solution (10%) and EtOAc. The aqueous phase was additionally extracted with EtOAc and the combined organic layers were washed with water, dried over Na₂SO₄, filtered and concentrated to dryness. The residue was purified by flash chromatography (silica gel, CH₂Cl₂/MeOH) to give the title compound (26 mg, 69% yield).

HPLC Rt (Method A): 1.69 min; ESI+−MS m/z: 412.2 (M+H)⁺.

This method was used for the preparation of examples 2-12 using suitable starting materials (and intermediates 1-6):

| | | | Rt | MS | HPLC |
|---|---|---|---|---|---|
| STRUCTURE | EX | CHEMICAL NAME | (min) | (M + H) | Method |
| 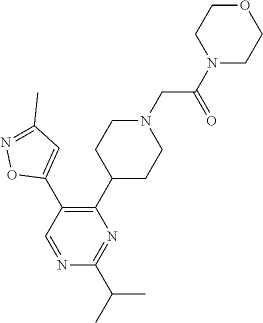 [SMILES: Cc1cc(-c2cnc(C(C)C)nc2C2CCN(CC(=O)N3CCOCC3)CC2)on1] | 2 | 2-(4-(2-Isopropyl-5- (3-methylisoxazol-5- yl)pyrimidin-4- yl)piperidin-1-yl)-1- morpholinoethanone | 1.78 | 414.2 | A |
| 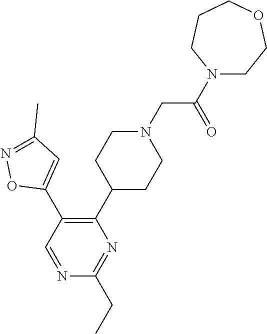 [SMILES: CCc1ncc(-c2cc(C)no2)c(C2CCN(CC(=O)N3CCCOCC3)CC2)n1] | 3 | 2-(4-(2-Ethyl-5-(3- methylisoxazol-5- yl)pyrimidin-4- yl)piperidin-1-yl)- 1-(1,4-oxazepan-4- yl)ethenone | 1.58 | 414.2 | A |
| 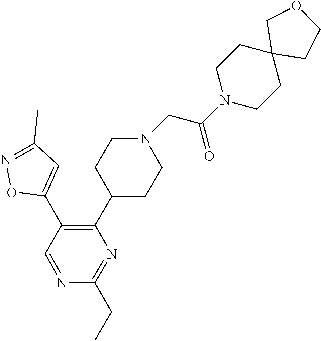 [SMILES: CCc1ncc(-c2cc(C)no2)c(C2CCN(CC(=O)N3CCC4(CCOC4)CC3)CC2)n1] | 4 | 2-(4-(2-Ethyl-5-(3- methylisoxazol-5- yl)pyrimidin-4- yl)piperidin-1-yl)- 1-(2-oxa-8- azaspiro[4.5]decan- 8-yl)ethenone | 1.71 | 454.2 | A |
| 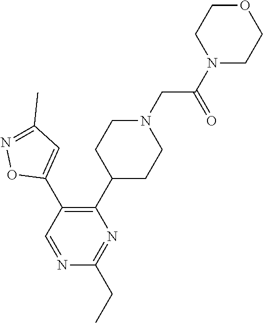 [SMILES: CCc1ncc(-c2cc(C)no2)c(C2CCN(CC(=O)N3CCOCC3)CC2)n1] | 5 | 2-(4-(2-Ethyl-5-(3- methylisoxazol-5- yl)pyrimidin-4- yl)piperidin-1-yl)-1- morpholinoethanone | 1.56 | 400.2 | A |
| 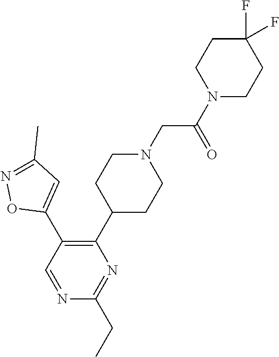 [SMILES: CCc1ncc(-c2cc(C)no2)c(C2CCN(CC(=O)N3CCC(F)(F)CC3)CC2)n1] | 6 | 1-(4,4- Difluoropiperidin- 1-yl)-2-(4-(2-ethyl- 5-(3-methylisoxazol- 5-yl)pyrimidin-4- yl)piperidin-1- yl)ethenone | 1.95 | 434.2 | A |
| 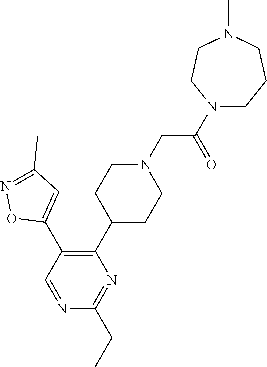 [SMILES: CCc1ncc(-c2cc(C)no2)c(C2CCN(CC(=O)N3CCCN(C)CC3)CC2)n1] | 7 | 2-(4-(2-Ethyl-5-(3- methylisoxazol-5- yl)pyrimidin-4- yl)piperidin-1-yl)-1- 4-methyl-1,4- diazepan-1- yl)ethenone | 1.62 | 427.2 | A |
| 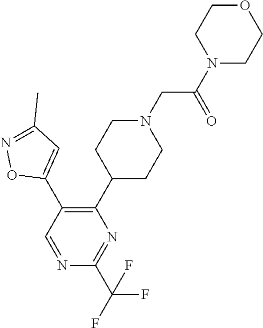 [SMILES: Cc1cc(-c2cnc(C(F)(F)F)nc2C2CCN(CC(=O)N3CCOCC3)CC2)on1] | 8 | 2-(4-(5-(3- Methylisoxazol-5- yl)-2- (trifluoromethyl)py- rimidin-4- yl)piperidin-1-yl)-1- morpholinoethanone | 1.80 | 440.2 | A |
| 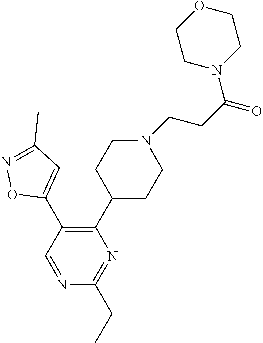 [SMILES: CCc1ncc(-c2cc(C)no2)c(C2CCN(CCC(=O)N3CCOCC3)CC2)n1] | 9 | 3-(4-(2-Ethyl-5-(3- methylisoxazol-5- yl)pyrimidin-4- yl)piperidin-1-yl)-1- morpholinopropan- 1-one | 1.44 | 414.2 | A |
| 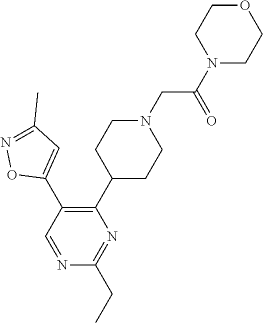 [SMILES: CCc1ncc(-c2cc(C)no2)c(C2CCN(CC(=O)N3CCOCC3)CC2)n1] | 10 | 4-(2-(4-(2-Ethyl-5- (3-methylisoxazol- 5-yl)pyrimidin-4- yl)piperidin-1- yl)ethyl)morpholine | 1.49 | 386.2 | A |
| 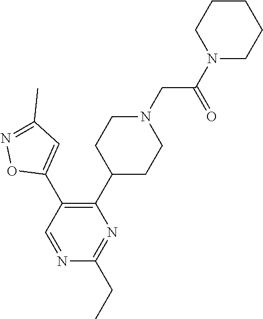 [SMILES: CCc1ncc(-c2cc(C)no2)c(C2CCN(CC(=O)N3CCCCC3)CC2)n1] | 11 | 2-(4-(2-Ethyl-5-(3- methylisoxazol-5- yl)pyrimidin-4- yl)piperidin-1-yl)- 1-(piperidin-1- yl)ethan-1-one | 1.93 | 398.2 | A |
| 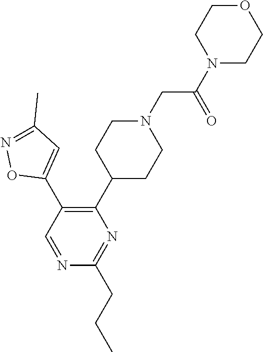 [SMILES: CCCc1ncc(-c2cc(C)no2)c(C2CCN(CC(=O)N3CCOCC3)CC2)n1] | 12 | 2-(4-(5-(3- Methylisoxazol- 5-yl)-2- propylpyrimidin-4- yl)piperidin-1-yl)- 1-morpholinoethan- 1-one | 1.72 | 414.2 | A |
| 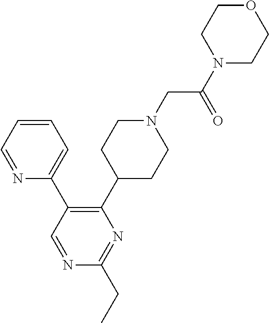 [SMILES: CCc1ncc(-c2ccccn2)c(C2CCN(CC(=O)N3CCOCC3)CC2)n1] | 13 | 2-(4-(2-Ethyl-5- (pyridin-2- yl)pyrimidin-4- yl)piperidin-1-yl)- 1-morpholinoethan- 1-one | 1.14 | 396.2 | A |
| 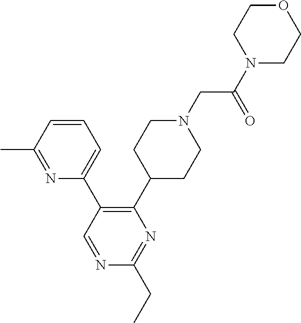 [SMILES: CCc1ncc(-c2cccc(C)n2)c(C2CCN(CC(=O)N3CCOCC3)CC2)n1] | 14 | 2-(4-(2-Ethyl-5-(6- methylpyridin-2- yl)pyrimidin-4- yl)piperidin-1-yl)- 1-morpholinoethan- 1-one | 1.53 | 410.2 | A |
| 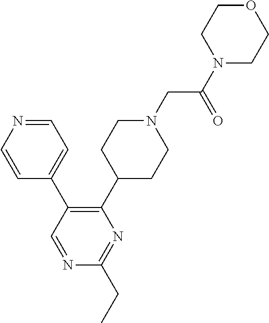 [SMILES: CCc1ncc(-c2ccncc2)c(C2CCN(CC(=O)N3CCOCC3)CC2)n1] | 15 | 2-(4-(2-Ethyl-5- (pyridin-4- yl)pyrimidin-4- yl)piperidin-1-yl)- 1-morpholinoethan- 1-one | 1.36 | 396.4 | A |
| 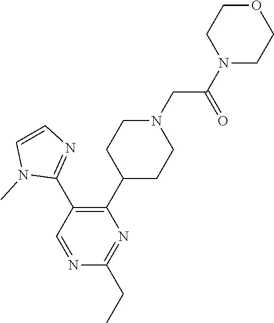 [SMILES: CCc1ncc(-c2nccn2C)c(C2CCN(CC(=O)N3CCOCC3)CC2)n1] | 16 | 2-(4-(2-Ethyl-5-(1- methyl-1H-imidazol- 2-yl)pyrimidin-4- yl)piperidin-1-yl)-1- morpholinoethan-1- one | 1.26 | 399.2 | A |

### Examples 17 and 18. (R)-2-(4-(2-Ethyl-5-(3-methylisoxazol-5-yl)pyrimidin-4-yl)azepan-1-yl)-1-morpholinoethan-1-one and (S)-2-(4-(2-ethyl-5-(3-methylisoxazol-5-yl)pyrimidin-4-yl)azepan-1-yl)-1-morpholinoethan-1-one.

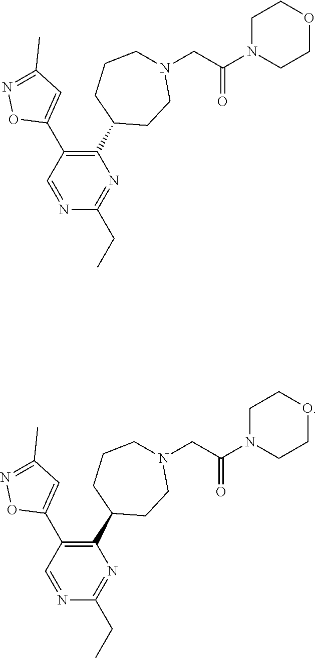 [SMILES: CCc1ncc(-c2cc(C)no2)c([C@@H]2CCCN(CC(=O)N3CCOCC3)CC2)n1 ; CCc1ncc(-c2cc(C)no2)c([C@H]2CCCN(CC(=O)N3CCOCC3)CC2)n1]

Starting from 2-(4-(2-ethyl-5-(3-methylisoxazol-5-yl)pyrimidin-4-yl)azepan-1-yl)-1-morpholinoethan-1-one, obtained following the procedure described in example 1, a chiral preparative HPLC separation was carried out to give the title compounds. Column Chiralpak IG 20×250 mm, 5 μm; temperature: r.t .; eluent: n-Heptane/EtOH/Et₂NH 90/0/0.03 v/v/v; flow rate 15 mL/min; Rt1: 98 min; Rt2: 110 min.

HPLC Rt (Method A): 1.69 min; ESI+−MS m/z: 414.2 (M+H)⁺.

### Example 19. 2-(4-(6-Methyl-3-(2-methylpyrimidin-4-yl)pyridin-2-yl)piperidin-1-yl)-1-morpholinoethan-1-one.

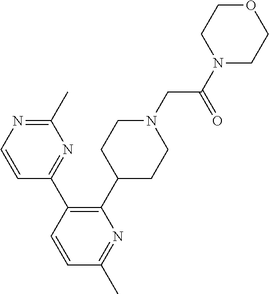 [SMILES: Cc1ccc(-c2ccnc(C)n2)c(C2CCN(CC(=O)N3CCOCC3)CC2)n1]

Step 1. 4-(2-Chloro-6-methylpyridin-3-yl)-2-methylpyrimidine.

A schlenk flask was charged with 4-chloro-2-methylpyrimidine (50 mg, 0.39 mmol), (2-chloro-6-methylpyridin-3-yl)boronic acid (80 mg, 0.47 mmol), Cs₂CO₃ (304 mg, 0.93 mmol) and Pd(PPh₃)₄ (90 mg, 0.08 mmol) and was evacuated and backfilled with argon. Dioxane:water (3:1) (2 mL), previously degassed by means of bubbling argon for 5 min, was added and the reaction mixture was stirred at 80° C. overnight. All volatiles were removed under reduced pressure and the residue was directly purified by flash chromatography (silica gel, Chx/EtOAc) to give the title compound (51 mg, 60% yield).

HPLC Rt (Method A): 0.90 min; ESI+−MS m/z: 272.2 (M+H)⁺.

Step 2. 1′-Benzyl-6-methyl-3-(2-methylpyrimidin-4-yl)-1′,2′,3′,6′-tetrahydro-2,4′-bipyridine.

A schlenk flask was charged with the product obtained in step 1 (55 mg, 0.25 mmol), 1-benzyl-4-(4,4,5,5-tetramethyl-1,3,2-dioxaborolan-2-yl)-1,2,3,6-tetrahydropyridine (90 mg, 0.3 mmol), Na2CO₃ (80 mg, 0.75 mmol) and Pd(PPh3)4 (29 mg, 0.025 mmol) and was evacuated and backfilled with argon. Dioxane:water (3:1) (5 mL), previously degassed by means of bubbling argon for 5 min, was added and the reaction mixture was stirred at 90° C. overnight. All volatiles were removed under reduced pressure and the residue was partitioned between aq sat NaHCO₃ solution and EtOAc. The aqueous phase was additionally extracted with EtOAc and the combined organic layers were dried over Na₂SO₄, filtered and concentrated to dryness. The residue was purified by flash chromatography (silica gel, CH₂Cl₂/MeOH) to give the title compound (60 mg, 67% yield).

HPLC Rt (Method B): 1.83 min; ESI+−MS m/z: 357.1 (M+H)⁺.

Step 3. 4-(4,4,5,5-Tetramethyl-1,3,2-dioxaborolan-2-yl)piperidine.

To a solution of the product obtained in step 2 (60 mg, 0.17 mmol) in MeOH (10 mL), previously purged with nitrogen, palladium (10 wt % on charcoal, wet, 18 mg) was added. The reaction flask was purged with H₂ by bubbling it through the suspension. The reaction was stirred at rt for 16 h. The catalyst was filtered off over a pad of Celite and the filtrate was evaporated to dryness. The residue was submitted to a second reaction cycle. The residue was re-dissolved in TFE (10 mL) and palladium (10 wt % on charcoal, wet, 25 mg) was added. The reaction flask was purged with H₂ by bubbling it through the suspension. The reaction was stirred at rt for 16 h. The catalyst was filtered off over a pad of Celite and the filtrate was evaporated to dryness to give the title compound (35 mg (36% purity), 28% yield).

HPLC Rt (Method): 1.12 min; ESI+−MS m/z: 269.2 (M+H)⁺.

### Step 4. Title Compound.

Starting from the product obtained in step 3 (35 mg (36% purity), 0.047 mmol) and following the experimental procedure described in step 5 of Example 1, the title compound was obtained (5 mg, 27% yield).

HPLC Rt (Method A): 1.36 min; ESI+−MS m/z: 396.2 (M+H)⁺.

### Biological Activity

### Pharmacological Study

This invention is aimed at providing a series of compounds which show pharmacological activity towards the σ₁ receptor and/or σ₂ receptor and, especially, compounds which have a binding expressed as Kᵢ responding to the following scales:
Kᵢ (σ₁) is preferably<1000 nM, more preferably<500 nM, even more preferably<100 nM; and
Kᵢ (σ₂) is preferably<1000 nM, more preferably<500 nM, even more preferably<100 nM.

### Human σ₁ Receptor Radioligand Assay

Transfected HEK-293 membranes (7 μg) were incubated with 5 nM of [³H](+)-pentazocine in assay buffer containing Tris-HCl 50 mM at pH 8. NBS (non-specific binding) was measured by adding 10 μM haloperidol. The binding of the test compound was measured at either one concentration (% inhibition at 1 or 10 μM) or five different concentrations to determine affinity values (Kᵢ). Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

### Binding Assay to Human σ₂/TMEM97 Receptor

Transfected HEK-293 membranes (15 μg) were incubated with 10 nM [³H]-1,3-Di-o-tolylguanidine (DTG) in assay buffer containing Tris-HCl 50 mM at pH 8.0. NSB (non-specific binding) was measured by adding 10 μM haloperidol. The binding of the test compound was measured at either one concentration (% inhibition at 1 or 10 μM) or five different concentrations to determine affinity values (Kᵢ). Plates were incubated at 25° C. for 120 minutes. After the incubation period, the reaction mix was transferred to MultiScreen HTS, FC plates (Millipore), filtered and washed 3 times with ice-cold 10 mM Tris-HCL (pH 8.0). Filters were dried and counted at approximately 40% efficiency in a MicroBeta scintillation counter (Perkin-Elmer) using EcoScint liquid scintillation cocktail.

### Results:

The following scale has been adopted for representing the binding to σ₁-receptor expressed as Kᵢ:
+ Kᵢ(σ₁)>1000 nM or inhibition ranges between 1% and 50%.
++ 500 nM<=Kᵢ(σ₁)<=1000 nM
+++ 100 nM<=Kᵢ(σ₁)<=500 nM
++++ Kᵢ(σ₁)<100 nM

The following scale has been adopted for representing the binding to σ2-receptor expressed as Kᵢ:
+ Kᵢ(σ₂)>1000 nM or inhibition ranges between 1% and 50%.
++ 500 nM<=Kᵢ(σ₂)<=1000 nM
+++ 100 nM<=Kᵢ(σ₂)<=500 nM
++++ Kᵢ(σ₂)<100 nM

The results of the compounds showing binding for the σ-1 and/or σ-2 receptor are shown in Table 1:

TABLE 1

| Example | Binding σ-1 | Binding σ-2 |
|---|---|---|
| 1 | ++ | ++++ |
| 2 | + | ++++ |
| 3 | + | ++++ |
| 4 | + | ++++ |
| 5 | + | ++++ |
| 6 | ++ | ++++ |
| 7 | + | ++++ |
| 8 | + | ++++ |
| 9 | + | ++++ |
| 10 | +++ | ++++ |
| 11 | ++ | ++++ |
| 12 | + | ++++ |
| 13 | + | +++ |
| 14 | + | +++ |
| 15 | ++ | ++ |
| 16 | + | ++ |
| 17 | +++ | +++ |
| 18 | ++ | ++++ |
| 19 | ++ | ++ |

1-13. (canceled)

14. A compound of the formula: 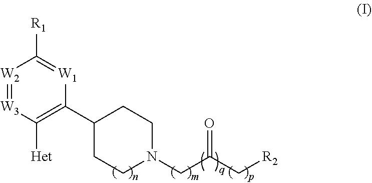
a stereoisomer thereof, a corresponding salt thereof, a co-crystal thereof, a prodrug thereof, or a solvate thereof,
wherein
each of m and n is independently 1 or 2;
each of p and q is independently 0 or 1;
each of W₁, W₂, W₃ is independently ═CH— or ═N—, provided at least one of W₁, W₂, or W₃ is ═N—
R₁ is a linear or branched C₁-C₆ alkyl; C₁-C₆ haloalkyl; or optionally substituted C₃-6 cycloalkyl;
Het is an optionally mono or polysubstituted C₃-C₉ heterocyclyl having at least one heteroatom selected from the group consisting of N, O or S;
R₂ is a moiety of the formula: 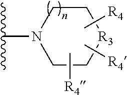
wherein
R₃ is selected from the group consisting of —C(R₄)(R₄′)—, —N(R₄)—, or —O—; and
each of R₄, R₄′ R₄″ is independently selected from the group consisting of H, C₁₋₆ alkyl, C₁₋₆ haloalkyl, C₁₋₆ alkoxy, —CN, —NRR′, wherein R and R′ are independently selected from the group consisting of H or C₁₋₆ alkyl;
or alternatively when R₄ and R₄′ are attached to the same carbon atom, together they can form a carbocyclic or heterocyclic spiro ring;
with the proviso that 2-[4-[2-methyl-5-(3-methyl-5-isoxazolyl)-4-pyrimidinyl]-1-piperidinyl]-1-(4-morpholinyl)-ethanone is excluded.

15. The compound according to claim 14, wherein R₁ is a C₁-C₆ alkyl, C₃-6 cycloalkyl, or C₁-6-haloalkyl.

16. The compound according to claim 15, wherein R₁ is ethyl, propyl, isopropyl, cyclopropyl, or trifluoromethyl.

17. The compound according to claim 14, wherein Het is an optionally monosubstituted isoxazole, an optionally monosubstituted pyrimidine, an optionally monosubstited pyridine, or an optionally monosubstituted imidazole.

18. The compound according to claim 14, wherein R₂ is selected from the group consisting of: 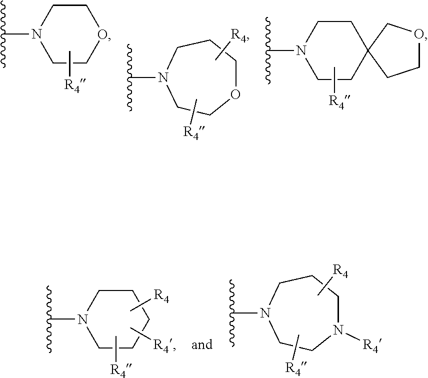
wherein R₄, R₄′, and R₄″ are as defined in claim 14.

19. The compound according to claim 14, wherein (i) W₃ is CH— and W₁ and W₂ are —N— or (ii) W₁ is —N— and W₂ and W₃ are —CH—.

20. The compound according to claim 14, wherein said compound is selected from the group consisting of:
2-(4-(2-Cyclopropyl-5-(3-methylisoxazol-5-yl)pyrimidin-4-yl)piperidin-1-yl)-1-morpholinoethanone;
2-(4-(2-Isopropyl-5-(3-methylisoxazol-5-yl)pyrimidin-4-yl)piperidin-1-yl)-1-morpholinoethanone;
2-(4-(2-Ethyl-5-(3-methylisoxazol-5-yl)pyrimidin-4-yl)piperidin-1-yl)-1-(1,4-oxazepan-4-yl)ethanone;
2-(4-(2-Ethyl-5-(3-methylisoxazol-5-yl)pyrimidin-4-yl)piperidin-1-yl)-1-(2-oxa-8-azaspiro[4.5]decan-8-yl)ethanone;
2-(4-(2-Ethyl-5-(3-methylisoxazol-5-yl)pyrimidin-4-yl)piperidin-1-yl)-1-morpholinoethanone;
1-(4,4-Difluoropiperidin-1-yl)-2-(4-(2-ethyl-5-(3-methylisoxazol-5-yl)pyrimidin-4-yl)piperidin-1-yl)ethanone;
2-(4-(2-Ethyl-5-(3-methylisoxazol-5-yl)pyrimidin-4-yl)piperidin-1-yl)-1-(4-methyl-1,4-diazepan-1-yl)ethanone;
2-(4-(5-(3-Methylisoxazol-5-yl)-2-(trifluoromethyl)pyrimidin-4-yl)piperidin-1-yl)-1-morpholinoethanone;
3-(4-(2-Ethyl-5-(3-methylisoxazol-5-yl)pyrimidin-4-yl)piperidin-1-yl)-1-morpholinopropan-1-one;
4-(2-(4-(2-Ethyl-5-(3-methylisoxazol-5-yl)pyrimidin-4-yl)piperidin-1-yl)ethyl)morpholine;
2-(4-(2-Ethyl-5-(3-methylisoxazol-5-yl)pyrimidin-4-yl)piperidin-1-yl)-1-(piperidin-1-yl)ethan-1-one;
2-(4-(5-(3-Methylisoxazol-5-yl)-2-propylpyrimidin-4-yl)piperidin-1-yl)-1-morpholinoethan-1-one;
2-(4-(2-Ethyl-5-(pyridin-2-yl)pyrimidin-4-yl)piperidin-1-yl)-1-morpholinoethan-1-one;
2-(4-(2-Ethyl-5-(6-methylpyridin-2-yl)pyrimidin-4-yl)piperidin-1-yl)-1-morpholinoethan-1-one;
2-(4-(2-Ethyl-5-(pyridin-4-yl)pyrimidin-4-yl)piperidin-1-yl)-1-morpholinoethan-1-one;
2-(4-(2-Ethyl-5-(1-methyl-1H-imidazol-2-yl)pyrimidin-4-yl)piperidin-1-yl)-1-morpholinoethan-1-one;
(R)-2-(4-(2-Ethyl-5-(3-methylisoxazol-5-yl)pyrimidin-4-yl)azepan-1-yl)-1-morpholinoethan-1-one;
(S)-2-(4-(2-Ethyl-5-(3-methylisoxazol-5-yl)pyrimidin-4-yl)azepan-1-yl)-1-morpholinoethan-1-one; and
2-(4-(6-Methyl-3-(2-methylpyrimidin-4-yl)pyridin-2-yl)piperidin-1-yl)-1-morpholinoethan-1-one.

21- A process for producing a compound of formula (I): 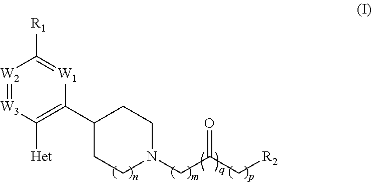
said method comprising contacting a compound of formula (V): 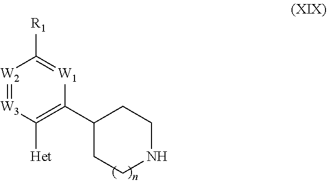
with a compound of formula (IX): 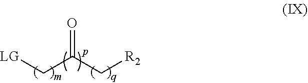
under conditions sufficient to produce the compound of formula (I),
wherein
each of m and n is independently 1 or 2;
each of p and q is independently 0 or 1;
each of W₁, W₂, W₃ is independently ═CH— or ═N—, provided at least one of W₁, W₂, or W₃ is ═N—
R₁ is a linear or branched C₁-C₆ alkyl; C₁-C₆ haloalkyl; or optionally substituted C₃-6 cycloalkyl;
Het is an optionally mono or polysubstituted C₃-C₉ heterocyclyl having at least one heteroatom selected from the group consisting of N, O or S;
R₂ is a moiety of the formula: 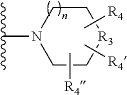
wherein
R₃ is selected from the group consisting of —C(R₄)(R₄′)—, —N(R₄)—, or —O—; and
each of R₄, R4′ R4″ is independently selected from the group consisting of H, C₁₋₆ alkyl, C₁₋₆ haloalkyl, C₁₋₆ alkoxy, —CN, —NRR′, wherein R and R′ are independently selected from the group consisting of H or C₁₋₆ alkyl;
or alternatively when R₄ and R₄′ are attached to the same carbon atom, together they can form a carbocyclic or heterocyclic spiro ring; and
LG is a leaving group.

22. The process according to claim 21, wherein W₃ is ═CH— and W₁ and W₂ are ═N—.

23. A method for treating a subject suffering from a clinical condition mediated by a sigma receptor, said method comprising administering a therapeutically effective amount of a compound of claim 14.

24. The method according to claim 23, wherein said sigma receptor is a sigma-1 receptor, a sigma-2 receptor, or a combination thereof.

25. The method according to claim 23, wherein said clinical condition comprises pain or a CNS disorder or disease.

26. The method according to claim 25, wherein said pain is selected from the group consisting of neuropathic pain, inflammatory pain, chronic pain, a pain condition involving allodynia or hyperalgesia.

27. The method according to claim 25, wherein said CNS disorder or disease is selected from the group consisting of:
addiction to a drugs or a chemical substance;
anxiety;
attention-deficit-/hyperactivity disorder (ADHD);
autism spectrum disorder;
catalepsy;
cognition disorder;
learning disorder;
memory impairment;
depression;
encephalitis;
epilepsy;
headache disorder;
insomnia;
locked-in-syndrome;
meningitis;
migraine;
multiple sclerosis (MS);
leukodystrophies;
amyotrophic lateral sclerosis (ALS);
myelopathy;
narcolepsy;
neurodegenerative disease;
traumatic brain injury;
Alzheimer disease;
Gaucher's disease;
Huntington disease;
Parkinson disease;
Tourette's syndrome;
psychotic condition;
bipolar disorder;
schizophrenia; and
paranoia.

28. The method according to claim 27, wherein said addiction to a drug or a chemical substance comprises addiction to cocaine, amphetamine, ethanol, nicotine, or a combination thereof.

29. A pharmaceutical composition comprising (i) a compound of claim 14, a pharmaceutically acceptable salt thereof, an isomer thereof, a co-crystal thereof, a prodrug thereof, a solvate thereof, or a combination thereof, and (ii) a pharmaceutically acceptable carrier, additive, adjuvant, vehicle, or a combination thereof.

30. The pharmaceutical composition according to claim 29, wherein said compound comprises an enantiomer mixture, a diastereomer mixture, or a racemic mixture.

31- The pharmaceutical composition according to claim 29, wherein said compound comprises an enantiomerically enriched mixture.

32. The pharmaceutical composition according to claim 29, wherein said compound comprises a diastereomerically enriched mixture.
